(19) United States
(12) Patent Application Publication
(10) Pub. No.: US 2021/0030815 A1
(43) Pub. Date: Feb. 4, 2021

(54) METHOD FOR SELECTING PROBIOTICS

(71) Applicant: WHITE DOG LABS, INC., New Castle, DE (US)

(72) Inventors: Bryan P. Tracy, Wilmington, DE (US); Shawn William Jones, Bear, DE (US); Alon Karpol, Tel Mond (IL); Aharon M. Eyal, Jerusalem (IL)

(21) Appl. No.: 17/067,782

(22) Filed: Oct. 12, 2020

Related U.S. Application Data
(63) Continuation-in-part of application No. PCT/US2019/027247, filed on Apr. 12, 2019.
(60) Provisional application No. 62/657,353, filed on Apr. 13, 2018.

(51) Int. Cl. A61K 35/742; A61K 35/744; A61K 35/747

(52) U.S. Cl. CPC A61K 35/742; A61K 35/747; A61K 35/744

(57) ABSTRACT

Provided is an improved method for selecting a probiotic microorganism composition, the method comprising providing a first consortium of microorganisms having Microorganism Composition A; providing a first host animal having a first microbiome having Microorganism Composition B; administering said first consortium to said first host animal, whereby said first microbiome is converted to a second microbiome having Microorganism Composition C; collecting a first sample of said second microbiome; and transferring at least a fraction of said first sample and/or said treated first sample onto and/or into a growth medium and incubating at a temperature between 15 degrees Celsius and 70 degrees Celsius to form first selected microorganism composition having Microorganism Composition D; wherein Microorganism Composition A of said first consortium comprises at least 50 different strains; wherein each of Compositions A and B; B and C and D and A differ from each other.

### CROSS-REFERENCE TO RELATED APPLICATIONS

This patent application is a continuation-in-part of PCT international application number PCT/US2019/027247, having an international filing date of Apr. 12, 2019, published as international publication number WO 2019/200271 A1, which is hereby incorporated by reference in its entirety; which claims benefit and priority from U.S. 62/657,353, filed on Apr. 13, 2018, hereby incorporated by reference in its entirety.

### BACKGROUND OF THE INVENTION

Microorganism selection is a key step in biotechnology, pharmaceuticals and agriculture industries directed to the isolation of new and improved strains. Such selection has high economic impact due to increased production yield, better substrate utilization, and faster process of isolating of new strains. Of particularly high importance for today's industry is the selection for microorganisms such as bacteria and yeasts. That is because new microorganisms can be used as novel probiotics for human and animals and in the production of pharmaceuticals.

### SUMMARY OF THE INVENTION

According to an embodiment, provided is an improved method for selecting a probiotic microorganism composition, the method comprising (i) providing a first consortium of microorganisms having Microorganism Composition A; (ii) optionally treating at least a fraction of said first consortium of microorganisms to form a treated first consortium of microorganisms; (iii) providing a first host animal, which first host animal comprises a first microbiome having Microorganism Composition B; (iv) administering said first consortium and/or said treated first consortium to said first host animal, whereby said first microbiome of said first host animal is converted to a second microbiome having Microorganism Composition C; (v) collecting a first sample of said second microbiome from said first host animal; (vi) optionally treating at least a fraction of said first sample of said second microbiome to form a treated first sample; and (vii) transferring at least a fraction of said first sample and/or at least a fraction of said treated first sample onto and/or into a growth medium and incubating at a temperature between 15 degrees Celsius and 70 degrees Celsius to form first selected microorganisms composition having Microorganism Composition D; wherein (a) Microorganism Composition A of said first consortium comprises at least So different strains; (b) Microorganism Composition A differs from Microorganism Composition B; (c) Microorganism composition B differs from Microorganism Composition C and (d) Microorganism Composition D differs from Microorganism Composition A.

According to an embodiment, the method further comprising administering to a first treated animal at least a fraction of said first selected microorganism comprising Microorganism Composition D. According to an embodiment, provided is a probiotics composition comprising said first selected microorganism composition comprising Microorganism Composition D.

According to an embodiment, said treating at least a fraction of said first consortium of microorganisms comprises (a) providing an organic liquid comprising at least 70% by weight hydrophobic solvent; (b) forming a multiple phase medium comprising a selected amount of said first consortium of microorganisms and a selected amount of said organic liquid; and (c) maintaining said multiple phase medium at a temperature between 15 degrees Celsius and 70 degrees Celsius for at least one minute, whereby a treated first consortium of microorganisms is formed.

According to an embodiment, said treating at least a fraction of said first sample of second microbiome comprises (a) providing an organic liquid comprising at least 70% by weight hydrophobic solvent; (b) forming a multiple phase medium comprising a selected amount of said first sample of second microbiome and a selected amount of said organic liquid; and (c) maintaining said multiple phase medium at a temperature between 15 degrees Celsius and 70 degrees Celsius at least one minute, whereby a treated first sample of second microbiome is formed.

According to an embodiment, said providing a first consortium of organisms having Microorganism Composition A comprises (a) providing a primary consortium of microorganisms having Microorganism Composition E; (b) optionally treating at least a fraction of said primary consortium of microorganisms to form a treated primary consortium of microorganisms; (c) providing a primary host animal, which primary host animal comprises a third microbiome having Microorganism Composition F; (d) administering said primary consortium of organisms and/or said treated primary consortium to said primary host animal, whereby said third microbiome of said primary host animal is converted to a fourth microbiome having Microorganism Composition G; (e) collecting a primary sample of said fourth microbiome from said primary host animal; (f) optionally treating at least a fraction of said primary sample of said fourth microorganism to form a treated primary sample; (g) and optionally transferring at least a fraction of said primary sample, at least a fraction of said treated primary sample onto and/or into a growth medium and incubating at a temperature between 15 degrees Celsius and 70 degrees Celsius to form said first consortium of organisms having Microorganism Composition A.

According to an embodiment, the method further comprises (a) providing a second host animal, which second host animal comprises a fifth microbiome having Microorganism Composition H; (b) administering said first selected microorganisms composition having Microorganism Composition D and/or first sample of said second microbiome to said second host animal, whereby the microorganism composition of said second host animal is converted to a sixth microbiome having Microorganism Composition I; (c) collecting a second sample of said sixth microbiome from said second host animal; and optionally (d) transferring at least a fraction of said second sample, onto and/or into a growth medium and incubating at a temperature between 15 degrees Celsius and 70 degrees Celsius to form a second selected microorganism composition comprising Microorganism Composition J. According to an embodiment, the method further comprises administering to a second treated animal at least a fraction of said second selected microorganism composition comprising Microorganism Composition J. According to an embodiment, provided is a probiotics composition comprising said second selected microorganism composition comprising Microorganism Composition J.

According to an embodiment said first host animal is selected from the group consisting of poultry, swine, fish, fin-fish, shellfish, dogs, cats, horses, cattle, other ruminants and humans. According to an embodiment the method further comprises monitoring the health and performance of said first host animal during and/or after said administering. According to an embodiment said first treated animal is selected from the group consisting of poultry, swine, fish, fin-fish, shellfish, dogs, cats, horses, cattle, other ruminants and humans. According to an embodiment said primary host animal is selected from the group consisting of poultry, swine, fish, fin-fish, shellfish, dogs, cats, horses, cattle, other ruminants and humans. According to an embodiment said method further comprises monitoring the health and performance of said primary host animal during and/or after said administering. According to an embodiment said second treated animal is selected from the group consisting of poultry, swine, fish, fin-fish, shellfish, dogs, cats, horses, cattle, other ruminants and humans.

According to an embodiment said Microorganism Composition A comprises at least one strain from the genus Clostridium, at least one strain from the genus Lactobacillus, at least one strain from the genus Enterococcus, at least one strains from the genus Streptococcus, and\or at least one strain from the genus Eubacterium. According to an embodiment said Microorganism Composition A has at least a portion characterized by at least one of producing butyric acid and/or lactic acid when grown on glucose, having an inhibitory effect on pathogens, being capable of forming spore and utilizing lactic acid.

According to an embodiment said treated first consortium comprises at least one of Clostridium butyricum, Clostridium tyrobutyricum, Clostridium leptum, Clostridium coccoides, Clostridium scindens, Clostridium hylemonae, Clostridium hathewayi, Clostridium symbiosum, Clostridium indolis, Clostridium oroticum, Clostridium celerecrescens, Clostridium sphenoides, Clostridium saccharoperbutylacetonicum, and Clostridium sporogenes. According to an embodiment said treated first consortium has at least a portion characterized by at least one of producing butyric acid and/or lactic acid when grown on glucose, having an inhibitory effect on pathogens, being capable of forming spore and utilizing lactic acid.

According to an embodiment said Microorganism Composition E comprises at least one strain from the genus Clostridium, at least one strain from the genus Lactobacillus, at least one strain from the genus Enterococcus, at least one strains from the genus Streptococcus, and\or at least one strain from the genus Eubacterium. According to an embodiment said Microorganism Composition E has at least a portion characterized by at least one of producing butyric acid and/or lactic acid when grown on glucose, having an inhibitory effect on pathogens, being capable of forming spore and utilizing lactic acid.

According to an embodiment said treated primary consortium comprises at least one of Clostridium butyricum, Clostridium tyrobutyricum, Clostridium saccharoperbutylacetonicum, and Clostridium sporogenes. According to an embodiment said treated primary consortium has at least a portion characterized by at least one of producing butyric acid and/or lactic acid when grown on glucose, having an inhibitory effect on pathogens, being capable of forming spore and utilizing lactic acid.

According to an embodiment said Microorganism Composition D comprises at least one strain from the genus Clostridium, and/or at least one strain from the genus Eubacterium. According to an embodiment said Microorganism Composition D has at least a portion characterized by at least one of producing butyric acid and/or lactic acid when grown on glucose, having an inhibitory effect on pathogens, being capable of forming spore and utilizing lactic acid. According to an embodiment said Microorganism Composition J comprises at least one strain from the genus Clostridium, and/or at least one strain from the genus Eubacterium. According to an embodiment at least a fraction of said Microorganism Composition J is characterized by at least one of producing butyric acid as the major metabolite when grown on glucose, having an inhibitory effect on pathogens, being capable of forming spore and utilizing lactic acid.

### BRIEF DESCRIPTION OF THE DRAWINGS

FIG. 1 shows an exemplary process for selecting probiotics (100) in the form of a flow chart. Process steps (101), (102), (104), (106), (108), (100), and (112) resulting in microorganism compositions as set forth in (114) of the flow chart correspond to features (i)-(xi) as described in the Summary of the Invention section of this application.

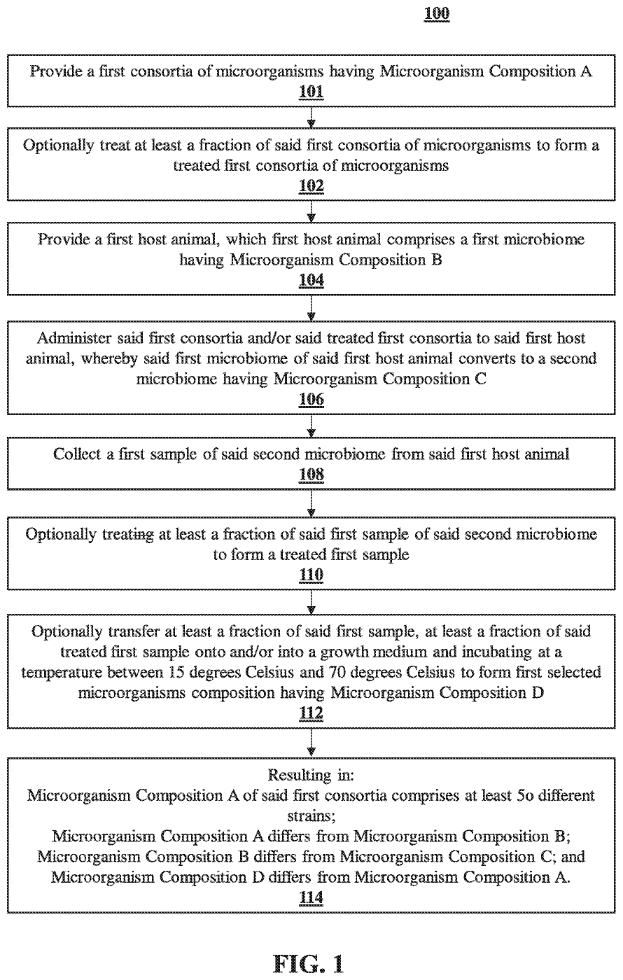

FIG. 2 shows further steps that could be performed in a second exemplary process for selecting probiotics (200) which steps include further providing a second host animal comprising a microbiome having Microorganism Composition FI (210), administering a selected microorganisms composition to the second host animal, whereby the microorganism composition of said second host animal converts to a microbiome having Microorganism Composition I (220), collecting a sample of said microbiome having Microorganism Composition I from said second host animal (230), and optionally transferring at least a fraction of said sample to a growth medium and incubating it to form a selected microorganisms composition comprising Microorganism Composition J (240).

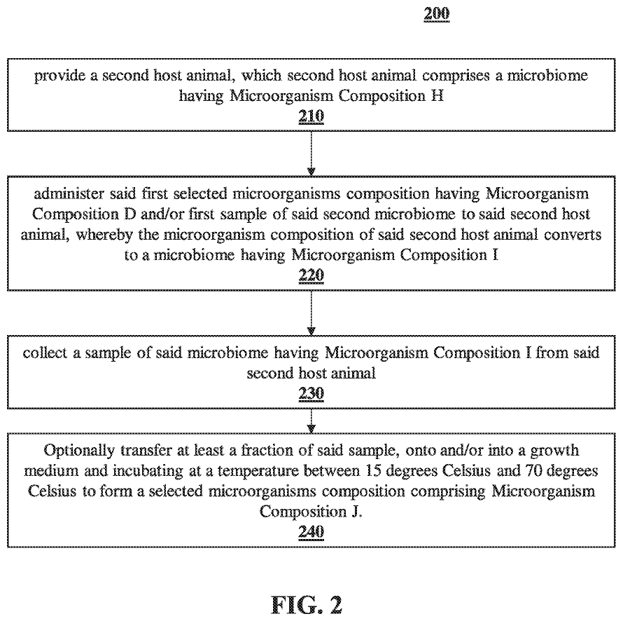

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

As used herein, the term “probiotic” refers to a live microorganism which provides health benefits to an animal when consumed, generally by restoring the balance of gut flora.

As used herein, the term “consortium” (or “consortia”) refers to two or more microbial groups living symbiotically.

As used herein, the term “microbiome” refers to an aggregate of all the microorganisms, symbiotic and pathogenic, living in and on an animal.

As used herein, the term “having an inhibitory effect on pathogens” with regard to a microorganism composition or consortium is intended to mean causing a reduction of at least 10% in the number of pathogens present in an animal as compared to number of pathogens present in the animal in the absence of use of the composition or consortium.

As used herein, the term “treating an animal in need thereof” is intended to mean preventing, curing, ameliorating, mitigating, and reducing the instances or severity of a condition or a symptom thereof in an animal in need thereof.

As used herein, the term “treating” with regard to a fraction of a consortium of microorganisms or a sample of a microbiome is intended to mean subjecting the fraction or the sample to specific chemical or physical treatment, such as contacting with an organic liquid; forming a multiple phase medium; and maintaining said multiple phase medium at a specified temperature for a specified duration.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description, which follows, and in part will be obvious from the description, or may be learned by practice of the invention. It is to be understood that both the foregoing general description and the following detailed description are exemplary and explanatory only and are not restrictive of the invention, as claimed.

According to an embodiment, provided is an improved method for selecting probiotics comprising (i) providing a first consortia of microorganisms having Microorganism Composition A; (ii) optionally treating at least a fraction of said first consortia of microorganisms to form a treated first consortia of microorganisms; (iii) providing a first host animal, which first host animal comprises a first microbiome having Microorganism Composition B; (iv) administering said first consortia and/or said treated first consortia to said first host animal, whereby said first microbiome of said first host animal converts to a second microbiome having Microorganism Composition C; (v) collecting a first sample of said second microbiome from said first host animal; (vi) optionally treating at least a fraction of said first sample of said second microbiome to form a treated first sample; and (vii) optionally transferring at least a fraction of said first sample, at least a fraction of said treated first sample onto and/or into a growth medium and incubating at a temperature between 15 degrees Celsius and 70 degrees Celsius to form first selected microorganisms composition having Microorganism Composition D; wherein (a) Microorganism Composition A of first consortia comprises at least So different strains; (b) Microorganism Composition A differs from Microorganism Composition B; (c) Microorganism composition B differs from Microorganism Composition C and (d) Microorganism Composition D differs from Microorganism Composition A.

According to an embodiment, said first consortia comprises at least 50 different strains, at least 100, at least 150 or at least 200 different strains. According to an embodiment, said first consortia comprises at least one strain from the genus Clostridium, at least one strain from the genus Lactobacillus, at least one strain from the genus Enterococcus, at least one strains from the genus Streptococcus, and\or at least one strain from the genus Eubacterium. According to an embodiment, at least a fraction of said first consortia is characterized by at least one of producing butyric acid and/or lactic acid when grown on glucose, having an inhibitory effect on pathogens, being capable of forming spore and utilizing lactic acid.

According to an embodiment, said providing a first consortia of organisms having Microorganism Composition A comprises (a) providing a primary consortia of microorganisms having Microorganism Composition E; (b) optionally treating at least a fraction of said primary consortia of microorganisms to form a treated primary consortia of microorganisms; (c) providing a primary host animal, which primary host animal comprises a third microbiome having Microorganism Composition F; (d) administering said primary consortia of organisms and/or said treated primary consortia to said primary host animal, whereby said third microbiome of said primary host animal converts to a fourth microbiome having Microorganism Composition G; (e) collecting a primary sample of said fourth microbiome from said primary host animal; (f) optionally treating at least a fraction of said primary sample of said fourth microorganism to form a treated primary sample; (g) and optionally transferring at least a fraction of said primary sample, at least a fraction of said treated primary sample onto and/or into a growth medium and incubating at a temperature between 15 degrees Celsius and 70 degrees Celsius to form said first consortia of organisms having Microorganism Composition A.

According to an embodiment, said primary consortia comprises at least So different strains, at least 100, at least 150 or at least 200 different strains. According to an embodiment, said primary consortia comprises at least one strain from the genus Clostridium, at least one strain from the genus Lactobacillus, at least one strain from the genus Enterococcus, at least one strains from the genus Streptococcus, and\or at least one strain from the genus Eubacterium. According to an embodiment, said primary consortia has at least a portion characterized by at least one of producing butyric acid and/or lactic acid when grown on glucose, having an inhibitory effect on pathogens, being capable of forming spore and utilizing lactic acid.

According to an embodiment, said treating at least a fraction of said primary consortia of microorganisms comprises (a) providing an organic liquid comprising at least 70% by weight hydrophobic solvent; (b) forming a multiple phase medium comprising a selected amount of said primary consortia of microorganisms and a selected amount of said organic liquid; and (c) maintaining said multiple phase medium at a temperature between 15 degrees Celsius and 70 degrees Celsius for at least one minute, whereby a treated primary consortia of microorganisms is formed.

According to an embodiment, said treating at least a fraction of said first consortia of microorganisms comprises (a) providing an organic liquid comprising at least 70% by weight hydrophobic solvent; (b) forming a multiple phase medium comprising a selected amount of said first consortia of microorganisms and a selected amount of said organic liquid; and (c) maintaining said multiple phase medium at a temperature between 15 degrees Celsius and 70 degrees Celsius for at least one minute, whereby a treated first consortia of microorganisms is formed.

According to an embodiment, said treating at least a fraction of said first sample of second microbiome comprises (a) providing an organic liquid comprising at least 70% by weight hydrophobic solvent; (b) forming a multiple phase medium comprising a selected amount of said first sample of second microbiome and a selected amount of said organic liquid; and (c) maintaining said multiple phase medium at a temperature between 15 degrees Celsius and 70 degrees Celsius at least one minute, whereby a treated first sample of second microbiome is formed.

According to an embodiment, said treating at least a fraction of said primary sample of said fourth microbiome comprises (a) providing an organic liquid comprising at least 70% by weight hydrophobic solvent; (b) forming a multiple phase medium comprising a selected amount of said primary sample of said fourth microbiome and a selected amount of said organic liquid; and (c) maintaining said multiple phase medium at a temperature between 15 degrees Celsius and 70 degrees Celsius at least one minute, whereby a treated primary sample of said fourth microbiome is formed.

According to an embodiment, said first host animal is selected from the group consisting of monogastric animals, poultry, swine, fish, fin-fish, shellfish, dogs, cats, horses, cattle, other ruminants and humans. According to an embodiment, said primary host animal is selected from the group consisting of monogastric animals, poultry, swine, fin-fish, shellfish, dogs, cats, and horses, humans. According to an embodiment, said first host animal is similar to said primary host animal, e.g. of the same family and/or similar age. According to an embodiment, said first host animal is different from said primary host animal, e.g. of a different family.

According to an embodiment, said administering said first consortia and/or said treated first consortia to said first host animal comprises oral administration with a feed, oral administration without a feed, administration in drink, administration in milk and/or via in-ovo injection. According to an embodiment, said administering said primary consortia and/or said treated primary consortia to said primary host animal comprises oral administration with a feed, oral administration without a feed, administration in drink, administration in milk and/or via in-ovo injection.

According to an embodiment, said treated first consortia of microorganisms comprises at least one of Clostridium butyricum, Clostridium tyrobutyricum, Clostridium leptum, Clostridium coccoides, Clostridium scindens, Clostridium hylemonae, Clostridium hathewayi, Clostridium symbiosum, Clostridium indolis, Clostridium oroticum, Clostridium celerecrescens, Clostridium sphenoides, Clostridium saccharoperbutylacetonicum, and Clostridium sporogenes. According to an embodiment, said treated first consortia of microorganisms has at least a portion characterized by at least one of producing butyric acid and/or lactic acid when grown on glucose, having an inhibitory effect on pathogens, being capable of forming spore and utilizing lactic acid. According to an embodiment, said Microorganism Composition B comprises at least one strain from the genus Clostridium, at least one strain from the genus Lactobacillus, at least one strain from the genus Enterococcus, at least one strains from the genus Streptococcus, and/or at least one strain from the genus Eubacterium. According to an embodiment, said Microorganism Composition B has at least a portion characterized by at least one of producing butyric acid and/or lactic acid when grown on glucose, having an inhibitory effect on pathogens, being capable of forming spore and utilizing lactic acid. According to an embodiment, said Microorganism Composition C comprises at least one strain from the genus Clostridium, at least one strain from the genus Lactobacillus, at least one strain from the genus Enterococcus, at least one strains from the genus Streptococcus, and/or at least one strain from the genus Eubacterium. According to an embodiment, said Microorganism Composition C has at least a portion characterized by at least one of producing butyric acid and/or lactic acid when grown on glucose, having an inhibitory effect on pathogens, being capable of forming spore and utilizing lactic acid. According to an embodiment, said Microorganism Composition D comprises at least one strain from the genus Clostridium, at least one strain from the genus Lactobacillus, at least one strain from the genus Enterococcus, at least one strains from the genus Streptococcus, and/or at least one strain from the genus Eubacterium. According to an embodiment, said Microorganism Composition D has at least a portion characterized by at least one of producing butyric acid and/or lactic acid when grown on glucose, having an inhibitory effect on pathogens, being capable of forming spore and utilizing lactic acid.

According to an embodiment, said treated primary consortia of microorganisms comprises at least one of Clostridium butyricum, Clostridium tyrobutyricum, Clostridium saccharoperbutylacetonicum, and Clostridium sporogenes. According to an embodiment said treated primary consortia has at least a portion characterized by at least one of producing butyric acid and/or lactic acid when grown on glucose, having an inhibitory effect on pathogens, being capable of forming spore and utilizing lactic acid.

According to an embodiment, said Microorganism Composition F comprises at least one strain from the genus Clostridium, at least one strain from the genus Lactobacillus, at least one strain from the genus Enterococcus, at least one strains from the genus Streptococcus, and/or at least one strain from the genus Eubacterium. According to an embodiment, said Microorganism Composition F has at least a portion characterized by at least one of producing butyric acid and/or lactic acid when grown on glucose, having an inhibitory effect on pathogens, being capable of forming spore and utilizing lactic acid. According to an embodiment, said Microorganism Composition G comprises at least one strain from the genus Clostridium, at least one strain from the genus Lactobacillus, at least one strain from the genus Enterococcus, at least one strains from the genus Streptococcus, and/or at least one strain from the genus Eubacterium. According to an embodiment, said Microorganism Composition G has at least a portion characterized by at least one of producing butyric acid and/or lactic acid when grown on glucose, having an inhibitory effect on pathogens, being capable of forming spore and utilizing lactic acid.

According to an embodiment, said collecting a first sample of said second microbiome from said first host animal comprises collecting a stool sample, harvesting material from the gastrointestinal track and/or harvesting part of the gastrointestinal track According to an embodiment, said method comprises administering said first consortia and/or said treated first consortia to multiple individuals of said first host animal, e.g. to multiple birds, and monitoring the health and performance of those first host individuals. According to an embodiment, said method comprises collecting said first sample of said second microbiome from particular individuals of said first host animals showing better health and/or performance. According to an embodiment, said better health and/or performance comprises at least one of greater weight gain, lower rate of infection, better resistance to pathogens, healthier epithelial cells, high titer of antibodies, and better immune response.

According to an embodiment, said collecting a primary sample of said fourth microbiome from said primary host animal comprises collecting a stool sample, harvesting material from the gastrointestinal track and/or harvesting part of the gastrointestinal track. According to an embodiment, said method comprises administering said primary consortia and/or said treated primary consortia to multiple individuals of said primary host animal, e.g. to multiple birds, and monitoring the health and performance of those primary host individuals. According to an embodiment, said method comprises collecting said first sample of said fourth microbiome from particular individuals of said primary host animal showing better health and/or performance. According to an embodiment, said better health and/or performance comprises at least one of greater weight gain, lower rate of infection, better resistance to pathogens, healthier epithelial cells, high titer of antibodies, and better immune response.

According to an embodiment, said method comprises transferring at least a fraction of said first sample, at least a fraction of said treated first sample onto and/or into a growth medium and incubating at a temperature between 15 degrees Celsius and 70 degrees Celsius to form first selected microorganism's composition having Microorganism Composition D.

According to an embodiment, said method comprises transferring at least a fraction of said primary sample, at least a fraction of said treated primary sample onto and/or into a growth medium and incubating at a temperature between 15 degrees Celsius and 70 degrees Celsius to form said first consortia of organisms having Microorganism Composition A.

According to an embodiment, said Microorganism Composition A differs from said Microorganism Composition B. According to an embodiment, said Microorganism composition B differs from said Microorganism Composition C. According to an embodiment, said Microorganism Composition D differs from Microorganism Composition A. According to an embodiment, said Microorganism Composition C, said Microorganism Composition D or both differ from said Microorganism Composition A, said Microorganism Composition B or both in at least one of better production of butyric acid and/or lactic acid when grown on glucose, having a better inhibitory effect on pathogens, having more Clostridium strains, being more capable of forming spore and better utilizing lactic acid.

According to an embodiment, said Microorganism Composition E differs from said Microorganism Composition F. According to an embodiment, said Microorganism composition F differs from said Microorganism Composition G. According to an embodiment, said Microorganism Composition G differs from Microorganism Composition A. According to an embodiment, said Microorganism Composition G differs from said Microorganism Composition E, said Microorganism Composition F or both in at least one of better production of butyric acid and/or lactic acid when grown on glucose, having a better inhibitory effect on pathogens, having more Clostridium strains, being more capable of forming spore and better utilizing lactic acid.

According to an embodiment, said method comprises administering to a first treated animal at least a fraction of said first selected microorganism comprising Microorganism Composition D. According to an embodiment, said first treated animal is selected from the group consisting of monogastric animals, poultry, swine, fish, fin-fish, shellfish, dogs, cats, horses, cattle, other ruminants and humans.

According to an embodiment, provided is probiotics comprising said first selected microorganism composition comprising Microorganism Composition D. According to an embodiment, said probiotics further comprises at least one of a strain that produces butyric acid, a strain that sporulate and a strain that has an inhibitory effect on pathogens.

According to an embodiment, said method, further comprising (a) providing a second host animal, which second host animal comprises a fifth microbiome having Microorganism Composition H; (b) administering said first selected microorganisms composition having Microorganism Composition D and/or first sample of said second microbiome to said second host animal, whereby the microorganism composition of said second host animal converts to a sixth microbiome having Microorganism Composition I; (c) collecting a second sample of said sixth microbiome from said second host animal; and optionally (d) transferring at least a fraction of said second sample, onto and/or into a growth medium and incubating at a temperature between 15 degrees Celsius and 70 degrees Celsius to form a second selected microorganism composition comprising Microorganism Composition J.

According to an embodiment, said second host animal is selected from the group consisting of monogastric animals, poultry, swine, fish, fin-fish, shellfish, dogs, cats, horses, cattle, other ruminants and humans. According to an embodiment, said second host animal is similar to said primary host animal. According to an embodiment, said first host animal is different from said primary host animal. According to an embodiment, said second host animal is similar to said first host animal. According to an embodiment, said first host animal is different from said first host animal. According to an embodiment, said administering to said second host animal comprises oral administration with a feed, oral administration without a feed, administration in drink, administration in milk and/or via in-ovo injection.

According to an embodiment, said collecting a second sample of said sixth microbiome from said second host animal comprises collecting a stool sample, harvesting material from the gastrointestinal track and/or harvesting part of the gastrointestinal track. According to an embodiment, said method comprises administering said first selected microorganism's composition having Microorganism Composition D and/or first sample of said second microbiome to multiple individuals of said second host animal, e.g. to multiple birds, and monitoring the health and performance of those second host individuals. According to an embodiment, said method comprises collecting said second sample of said sixth microbiome from particular individuals of said second host animal showing better health and/or performance. According to an embodiment, said better health and/or performance comprises at least one of greater weight gain, lower rate of infection, better resistance to pathogens, healthier epithelial cells, high titer of antibodies, and better immune response.

According to an embodiment, said Microorganism Composition I comprises at least one strain from the genus Clostridium, at least one strain from the genus Lactobacillus, at least one strain from the genus Enterococcus, at least one strains from the genus Streptococcus, and\or at least one strain from the genus Eubacterium. According to an embodiment, said Microorganism Composition J has at least a portion characterized by at least one of producing butyric acid and/or lactic acid when grown on glucose, having an inhibitory effect on pathogens, being capable of forming spore and utilizing lactic acid.

According to an embodiment, said Microorganism Composition H differs from said Microorganism Composition I. According to an embodiment, said Microorganism composition I differs from said Microorganism Composition J. According to an embodiment, said Microorganism Composition I, said Microorganism Composition J or both differ from said Microorganism Composition H in at least one of better production of butyric acid and/or lactic acid when grown on glucose, having a better inhibitory effect on pathogens, having more Clostridium strains, being more capable of forming spore and better utilizing lactic acid. According to an embodiment, said Microorganism Composition J differs from Microorganism Composition A. According to an embodiment, said Microorganism Composition I, said Microorganism Composition J or both differ from said Microorganism Composition A in at least one of better production of butyric acid and/or lactic acid when grown on glucose, having a better inhibitory effect on pathogens, having more Clostridium strains, being more capable of forming spore and better utilizing lactic acid.

According to an embodiment, said method comprises transferring at least a fraction of said second sample, onto and/or into a growth medium and incubating at a temperature between 15 degrees Celsius and 70 degrees Celsius to form a second selected microorganism composition comprising Microorganism Composition J.

According to an embodiment, said method comprises administering to a second treated animal at least a fraction of said second selected microorganism composition comprising Microorganism Composition J. According to an embodiment, said second treated animal is selected from the group consisting of monogastric animals, poultry, swine, fish, fin-fish, shellfish, dogs, cats, horses, cattle, other ruminants and human

According to an embodiment, further provided is probiotics comprising said second selected microorganism composition comprising Microorganism Composition J. According to an embodiment, said probiotics further comprises at least one of a strain that produces butyric acid, a strain that sporulate and a strain that has an inhibitory effect on pathogens.

### EXAMPLES

### Example 1. Spore Treatment of Same Animal Species

A fecal sample is collected from a healthy chicken. The healthy chicken has a microbiome consisting of many different bacteria (both in genus and species). The sample is mixed in sterile water (20% g/mL) for 16 hours at 15° C. at 100 rpm, and then passed through a 25 μm filter. The filtrate is mixed with 99.8% chloroform (50:50 ratio) and mixed end-over-end for 10 minutes at room temperature. The upper aqueous phase is removed and used to inoculate Reinforced Clostridial Medium (RCM) (10 g/L peptone, 10 g/L beef extract, 3 g/L yeast extract, 5 g/L dextrose, 5 g/L sodium chloride, 1 g/L soluble starch, 0.5 g/L cysteine HCl, 3 g/L sodium acetate, and 0.5 g/L agar). The RCM is incubated under anaerobic conditions at 37° C. until spores are formed (>3 days). The cell mass is then harvested, washed in a saline solution, and resuspended in a volume to yield a concentration factor of at least 25×. This concentrated cell mass consists of primarily Clostridium species and contains at least 100 different species.

The concentrated cell mass is then mixed with a feed and fed to an unhealthy chicken, for example, a chicken with a Clostridium perfringens infection. The unhealthy chicken has a microbiome consisting of many different bacteria (both in genus and species) but may be deficient in certain Clostridium species. The unhealthy chicken is fed the feed with the concentrated cell mass until its health improves, for example the C. perfringens infection is cured. A fecal sample is collected from the chicken with improved health. The chicken with an improved health has a microbiome consisting of many different bacteria (both in genus and species), and the composition of the microbiome is changed by introducing the concentrated cell mass. The fecal sample is mixed in sterile water (20% g/mL) for 16 hours at 15° C. at 100 rpm, and then passed through a 25 μm filter. The filtrate is mixed with 99.8% chloroform (50:50 ratio) and mixed end-over-end for 10 minutes at room temperature. The upper aqueous phase is removed and used to inoculate Reinforced Clostridial Medium (RCM). The RCM is incubated under anaerobic conditions at 37° C. until spores are formed (>3 days). The cell mass is then harvested, washed in a saline solution, and resuspended in a volume to yield a concentration factor of at least 25×. This concentrated cell mass consists of primarily Clostridium species and contains at least 100 different species. This concentrated, sporulated cell mass can be used to treat another unhealthy chicken, for example to help combat a C. perfringens infection.

### Example 2. Spore Treatment of Different Animal Species

A fecal sample is collected from a healthy chicken. The healthy chicken has a microbiome consisting of many different bacteria (both in genus and species). The sample is mixed in sterile water (20% g/mL) for 16 hours at 15° C. at 100 rpm, and then i passed through a 25 μm filter. The filtrate is mixed with 99.8% chloroform (50:50 ratio) and mixed end-over-end for 10 minutes at room temperature. The upper aqueous phase is removed and used to inoculate Reinforced Clostridial Medium (RCM) (10 g/L peptone, 10 g/L beef extract, 3 g/L yeast extract, 5 g/L dextrose, 5 g/L sodium chloride, 1 g/L soluble starch, 0.5 g/L cysteine HCl, 3 g/L sodium acetate, and 0.5 g/L agar). The RCM is incubated under anaerobic conditions at 37° C. until spores are formed (>3 days). The cell mass is then harvested, washed in a saline solution, and resuspended in a volume to yield a concentration factor of at least 25×. This concentrated cell mass consists of primarily Clostridium species and contains at least 100 different species.

The concentrated cell mass is then mixed with a feed and fed to a swine. The swine has a microbiome consisting of many different bacteria (both in genus and species). The swine is fed the feed with the concentrated cell mass until an improvement in health is observed, for example improved weight gain. A fecal sample is collected from the swine with improved health. The swine with an improved health has a microbiome consisting of many different bacteria (both in genus and species), and the microbiome is changed by introducing the concentrated cell mass. The fecal sample is mixed in sterile water (20% g/mL) for 16 hours at 15° C. at 100 rpm, and then passed through a 25 μm filter. The filtrate is mixed with 99.8% chloroform (50:50 ratio) and mixed end-over-end for 10 minutes at room temperature. The upper aqueous phase is removed and used to inoculate Reinforced Clostridial Medium (RCM). The RCM is incubated under anaerobic conditions at 37° C. until spores are formed (>3 days). The cell mass is then harvested, washed in a saline solution, and resuspended in a volume to yield a concentration factor of at least 25×. This cell mass consists of primarily Clostridium species and contains at least 100 different species. This concentrated, sporulated cell mass can be used to treat another swine or chicken to improve their health.

### Example 3. Treatment of Same Species

A fecal sample is collected from a healthy chicken. The healthy chicken has a microbiome consisting of many different bacteria (both in genus and species). The sample is mixed in sterile water (20% g/mL) for 16 hours at 15° C. at 100 rpm, and then passed through a 25 μm filter. The filtrate is used to inoculate Reinforced Clostridial Medium (RCM) (10 g/L peptone, 10 g/L beef extract, 3 g/L yeast extract, 5 g/L dextrose, 5 g/L sodium chloride, 1 g/L soluble starch, 0.5 g/L cysteine HCl, 3 g/L sodium acetate, and 0.5 g/L agar). The RCM is incubated under anaerobic conditions at 37° C. until spores are formed (>3 days). The cell mass is then harvested, washed in a saline solution, and resuspended in a volume to yield a concentration factor of at least 25×. This concentrated cell mass consists of many different anaerobic species and contains at least 100 different species.

The concentrated cell mass is then mixed with a feed and fed to an unhealthy chicken, for example, a chicken with a Clostridium perfringens infection. The unhealthy chicken has a microbiome consisting of many different bacteria (both in genus and species) but may be deficient in certain Clostridium species. The unhealthy chicken is fed the feed with the concentrated cell mass until its health improves, for example the C. perfringens infection is cured. A fecal sample is collected from the chicken with improved health. The chicken with an improved health has a microbiome consisting of many different bacteria (both in genus and species), and the composition is changed by introducing the concentrated cell mass. The fecal sample is mixed in sterile water (20% g/mL) for 16 hours at 15° C. at 100 rpm, and then it is passed through a 25 μm filter. The filtrate is used to inoculate Reinforced Clostridial Medium (RCM). The RCM is incubated under anaerobic conditions at 37° C. until spores are formed (>3 days). The cell mass is then harvested, washed in a saline solution, and resuspended in a volume to yield a concentration factor of at least 25×. This concentrated cell mass consists of many different anaerobic species and contains at least 100 different species. This concentrated, cell mass can be used to treat another chicken or other species.

1. An improved method for selecting a probiotic microorganism composition, the method comprising
(i) providing a first consortium of microorganisms having Microorganism Composition A;
(ii) optionally treating at least a fraction of said first consortium of microorganisms to form a treated first consortium of microorganisms;
(iii) providing a first host animal, which first host animal comprises a first microbiome having Microorganism Composition B;
(iv) administering said first consortium and/or said treated first consortium to said first host animal, whereby said first microbiome of said first host animal is converted to a second microbiome having Microorganism Composition C;
(v) collecting a first sample of said second microbiome from said first host animal;
(vi) optionally treating at least a fraction of said first sample of said second microbiome to form a treated first sample; and
(vii) transferring at least a fraction of said first sample and/or at least a fraction of said treated first sample onto and/or into a growth medium and incubating at a temperature between 15 degrees Celsius and 70 degrees Celsius to form first selected microorganism composition having Microorganism Composition D;
wherein
(a) Microorganism Composition A of said first consortium comprises at least So different strains;
(b) Microorganism Composition A differs from Microorganism Composition B;
(c) Microorganism composition B differs from Microorganism Composition C and
(d) Microorganism Composition D differs from Microorganism Composition A.

2. The method of claim 1, wherein said treating at least a fraction of said first consortium of microorganisms comprises (a) providing an organic liquid comprising at least 70% by weight hydrophobic solvent; (b) forming a multiple phase medium comprising a selected amount of said first consortium of microorganisms and a selected amount of said organic liquid; and (c) maintaining said multiple phase medium at a temperature between 15 degrees Celsius and 70 degrees Celsius for at least one minute, whereby a treated first consortium of microorganisms is formed.

3. The method of claim 1, wherein said treating at least a fraction of said first sample of second microbiome comprises (a) providing an organic liquid comprising at least 70% by weight hydrophobic solvent; (b) forming a multiple phase medium comprising a selected amount of said first sample of second microbiome and a selected amount of said organic liquid; and (c) maintaining said multiple phase medium at a temperature between 15 degrees Celsius and 70 degrees Celsius at least one minute, whereby a treated first sample of second microbiome is formed.

4. The method of claim 1, wherein said providing a first consortium of organisms having Microorganism Composition A comprises (a) providing a primary consortium of microorganisms having Microorganism Composition E; (b) optionally treating at least a fraction of said primary consortium of microorganisms to form a treated primary consortium of microorganisms; (c) providing a primary host animal, which primary host animal comprises a third microbiome having Microorganism Composition F; (d) administering said primary consortium of organisms and/or said treated primary consortium to said primary host animal, whereby said third microbiome of said primary host animal is converted to a fourth microbiome having Microorganism Composition G; (e) collecting a primary sample of said fourth microbiome from said primary host animal; (f) optionally treating at least a fraction of said primary sample of said fourth microbiome to form a treated primary sample; (g) and optionally transferring at least a fraction of said primary sample and/or at least a fraction of said treated primary sample onto and/or into a growth medium and incubating at a temperature between 15 degrees Celsius and 70 degrees Celsius to form said first consortium of organisms having Microorganism Composition A.

5. The method of claim 1, further comprising (a) providing a second host animal, which second host animal comprises a fifth microbiome having Microorganism Composition H; (b) administering said first selected microorganisms composition having Microorganism Composition D and/or first sample of said second microbiome to said second host animal, whereby the microorganism composition of said second host animal is converted to a sixth microbiome having Microorganism Composition I; (c) collecting a second sample of said sixth microbiome from said second host animal; and optionally (d) transferring at least a fraction of said second sample onto and/or into a growth medium and incubating at a temperature between 15 degrees Celsius and 70 degrees Celsius to form a second selected microorganism composition comprising Microorganism Composition J.

6. The method of claim 1, wherein said Microorganism Composition A comprises at least one strain from the genus Clostridium, at least one strain from the genus Lactobacillus, at least one strain from the genus Enterococcus, at least one strains from the genus Streptococcus, and\or at least one strain from the genus Eubacterium.

7. The method of claim 1, wherein said Microorganism Composition A has at least a portion characterized by at least one of producing butyric acid and/or lactic acid when grown on glucose, having an inhibitory effect on pathogens, being capable of forming spore and utilizing lactic acid.

8. The method of claim 1, wherein said treated first consortium comprises at least one of Clostridium butyricum, Clostridium tyrobutyricum, Clostridium leptum, Clostridium coccoides, Clostridium scindens, Clostridium hylemonae, Clostridium hathewayi, Clostridium symbiosum, Clostridium indolis, Clostridium oroticum, Clostridium celerecrescens, Clostridium sphenoides, Clostridium saccharoperbutylacetonicum, and Clostridium sporogenes.

9. The method of claim 1, wherein at least a portion of said treated first consortium is characterized by at least one of producing butyric acid as the major metabolite when grown on glucose, having an inhibitory effect on pathogens and being capable of forming spore and utilizing lactic acid.

10. The method of claim 4, wherein said Microorganism Composition E comprises at least one strain from the genus Clostridium, at least one strain from the genus Lactobacillus, at least one strain from the genus Enterococcus, at least one strains from the genus Streptococcus, and\or at least one strain from the genus Eubacterium.

11. The method of claim 4, wherein at least a portion of said Microorganism Composition E is characterized by at least one of producing butyric acid and/or lactic acid when grown on glucose, having an inhibitory effect on pathogens, being capable of forming spore and utilizing lactic acid.

12. The method of claim 4, wherein said treated primary consortium comprises at least one of Clostridium butyricum, Clostridium tyrobutyricum, Clostridium leptum, Clostridium coccoides, Clostridium scindens, Clostridium hylemonae, Clostridium hathewayi, Clostridium symbiosum, Clostridium indolis, Clostridium oroticum, Clostridium celerecrescens, Clostridium sphenoides, Clostridium saccharoperbutylacetonicum, and Clostridium sporogenes.

13. The method of claim 4, wherein said at least a fraction of said treated primary consortium is characterized by at least one of producing butyric acid as the major metabolite when grown on glucose, having an inhibitory effect on pathogens, being capable of forming spore and utilizing lactic acid.

14. The method of claim 1, wherein said Microorganism Composition D comprises at least one strain from the genus Clostridium, and/or at least one strain from the genus Eubacterium.

15. The method of claim 1, wherein at least a fraction of said Microorganism Composition D is characterized by at least one of producing butyric acid and/or lactic acid when grown on glucose, having an inhibitory effect on pathogens, being capable of forming spore and utilizing lactic acid.

16. The method of claim 5, wherein said Microorganism Composition J comprises at least one strain from the genus Clostridium, and/or at least one strain from the genus Eubacterium.

17. A probiotic composition comprising a first selected microorganism composition comprising Microorganism Composition D according to claim 1.

18. A method of treating an animal in need thereof, comprising administering to said animal at least a fraction of the probiotic composition Microorganism Composition of claim 16.

19. A probiotic composition comprising a second selected microorganism composition comprising Microorganism Composition J.

20. A method of treating an animal in need thereof, comprising administering to said animal at least a fraction of the probiotic composition Microorganism Composition of claim 18.
